(19) United States
(12) Patent Application Publication
(10) Pub. No.: US 2015/0250965 A1
(43) Pub. Date: Sep. 10, 2015

(54) Air Bubble

(71) Applicant: Felipe Kelly De Rivero, Oviedo, FL (US)

(72) Inventor: Felipe Kelly De Rivero, Oviedo, FL (US)

(21) Appl. No.: 14/199,413

(22) Filed: Mar. 6, 2014

(51) Int. Cl. A61M 16/00; A62B 9/04; A62B 7/12; A62B 9/06

(52) U.S. Cl. CPC A61M 16/0078; A62B 9/06; A62B 9/04; A62B 7/12

(57) ABSTRACT

Air Bubble is designed to extract one breath of air from under water via an air extraction backpack. The device includes an air-permeable extraction assembly that is attached to a backpack. The air-permeable extraction assembly has an air intake cavity connected to a durable impact resistant & underwater pressure resistant air bag with plastic reinforcing “ribs” inside bag, and a mouth piece that will release the air when pressure is applied onto the exterior walls. An air-permeable plastic cap will cover the air intake cavity. A mouthpiece draws intake air through the air intake cavity upon inhalation by a user. A transfer channel or tube passes intake air from the air-permeable extraction assembly to the mouthpiece when pressure is applied into exterior walls of mouth piece and upon inhalation by the user.

### BACKGROUND OF THE INVENTION

Backpack allows for one final air breath intake under water.

### BRIEF SUMMARY OF THE INVENTION

Air Bubble is designed to extract one breath of air from under water via an air extraction backpack. The device includes an air-permeable extraction assembly that is attached to a backpack. The air-permeable extraction assembly has an air intake cavity connected to a durable impact resistant & underwater pressure resistant air bag with plastic reinforcing “ribs” inside bag, and a mouth piece that will release the air when pressure is applied onto the exterior walls. An air-permeable plastic cap will cover the air intake cavity. A mouthpiece is in fluid communication with the air intake cavity. The mouthpiece draws intake air through the air intake cavity upon inhalation by a user. A transfer channel or tube passes intake air from the air-permeable extraction assembly to the mouthpiece when pressure is applied into exterior walls of mouth piece and upon inhalation by the user.

### BACKGROUND OF THE INVENTION

1. Field of the Invention

Embodiments of this invention relate to emergency breathing devices and more particularly relate to air containments for air extraction under water.

2. Description of the Related Art

In a typical water incident, water impacts user at high speed and individual may run out of air. Victim typically finds himself disoriented and out of air in water.

Item is intended for one use only during an emergency. If victim breathes from the bag several times, the oxygen is rapidly consumed and replaced by carbon dioxide. Within a short time, the lack of oxygen and/or the abundance of carbon dioxide may cause asphyxiation and/or suffocation.

Similar devices have been contrived in an attempt to extend the breathing time of a fully buried avalanche victim. Unfortunately, these devices do not address the problems resulting from being under water. Snow air bags are too heavy, bulky and they don't have a non-collapsible air bag. Other devices employ oxygen tanks in order to provide additional oxygen to the victim in addition to or in place of the diffused air. However, these devices are both bulky and heavy and, therefore, inhibit the movement of the user in many of the recreational activities that draw the users to the water in the first place. Similar backpack designs can be found for the purpose of drinking water or other liquids while exercising outdoors, but those bags serve a different purpose than our invention, they are for drinking liquids and ours is for inhaling one breath of air.

### BRIEF DESCRIPTION OF THE DRAWING

In order that the advantages of the invention will be readily understood, a more particular description of the invention briefly described above will be rendered by reference to specific embodiments that are illustrated in the drawing. Understanding that this drawing depict only typical embodiments of the invention and are not therefore to be considered to be limiting of its scope, the invention will be described and explained with additional specificity and detail through the use of the accompanying drawing, in which:

1. Mouth piece

2. Air transfer channel or tube

3. Compact shaped backpack

4. Water Permeable Non-Collapsible Bag

5. Carrying Adjustable Straps

6. Two Plastic Reinforcing Ribs with 3 evenly spaced holes each (inside the water permeable non-collapsible bag)

### DETAILED DESCRIPTION OF THE INVENTION

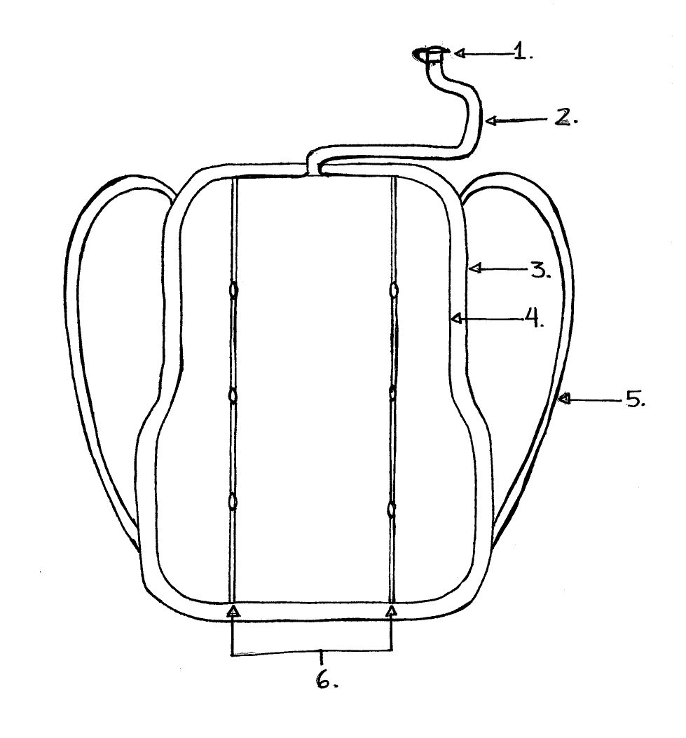

FIG. 1 depicts the Air Bubble backpack interior & exterior components.

1. Mouth piece—This plastic component will release the air when pressure is applied onto the exterior walls. An air-permeable plastic cap will cover the air intake cavity. A mouthpiece is in fluid communication with the air intake cavity. The mouthpiece draws intake air through the air intake cavity upon inhalation by a user.

2. Air transfer channel or tube—The component that connects the mouthpiece to the air bag allowing air to pass from the water permeable non collapsible bag to the mouthpiece upon inhalation

3. Compact shaped backpack—Fits tightly within user's shoulder blades and it has extra cushion to encapsulate and protect the water permeable non collapsible bag. Small enough to fit inside a wetsuit if preferred.

4. Water Permeable Non-Collapsible Bag—with Plastic Reinforcing Ribs to prevent bag from breaking/exploding under water.

5. Carrying Adjustable Straps—The carrying adjustable straps are designed to be worn on the shoulders of the user in a manner similar to other backpacks, such as full backpacks for school or compact sporting backpacks.

6. Inside the water permeable non-collapsible bag, two ribs have 3 evenly spaced holes each for airflow inside the bag.

1. An apparatus and system are disclosed to extract one breath of air from under water via an air extraction backpack. The device includes an air-permeable extraction assembly that is attached to a backpack. The air-permeable extraction assembly has an air intake cavity connected to a durable impact resistant & underwater pressure resistant air bag with plastic reinforcing “ribs” inside bag, and a mouth piece that will release the air when pressure is applied onto the exterior walls. An air-permeable plastic cap will cover the air intake cavity. A mouthpiece is in fluid communication with the air intake cavity. The mouthpiece draws intake air through the air intake cavity upon inhalation by a user. A transfer channel or tube passes intake air from the air-permeable extraction assembly to the mouthpiece when pressure is applied into exterior walls of mouth piece and upon inhalation by the user.

2. The apparatus of claim 1, further comprising a protective underwater resistant water-permeable non-collapsible bag preventing penetration of water into the air intake cavity. Plastic reinforcing ribs located in the interior of the air bag containment to provide impact resistance qualities and three evenly spaced holes each to allow air flow compartments within the bag.

3. The apparatus of claim 1, further comprising an air transfer channel (tube).

4. The apparatus of claim 1, further comprising an extraction assembly attachment device or mouth piece that will release the air when pressure is applied onto the exterior walls.

5. A compact shaped backpack; with two carrying strap attached to the backpack to allow a user to carry the backpack between shoulder blades. Small enough to fit inside a wetsuit if preferred. Bag to have a cushion to further aid with impact resistance.
